(19) United States
(12) Patent Application Publication
(10) Pub. No.: US 2024/0293507 A1
(43) Pub. Date: Sep. 5, 2024

(54) Methods for Enhancing Immune Checkpoint Inhibitor Therapy

(71) Applicant: The University of Southern California, Los Angeles, CA (US)

(72) Inventors: Michael E. SELSTED, Pasadena, CA (US); Dat Q. TRAN, Alhambra, CA (US); Jagadish KRISHNAPPA, Aracadia, CA (US)

(21) Appl. No.: 18/669,253

(22) Filed: May 20, 2024

Related U.S. Application Data
(63) Continuation of application No. PCT/US2022/080312, filed on Nov. 22, 2022.
(60) Provisional application No. 63/283,152, filed on Nov. 24, 2021.
(60) Provisional application No. 63/283,155, filed on Nov. 24, 2021.
(60) Provisional application No. 63/283,158, filed on Nov. 24, 2021.

(51) Int. Cl. A61K 38/17; A61K 39/395; A61P 35/00; C07K 7/64; C07K 14/47

(52) U.S. Cl. CPC A61K 38/1729; A61K 39/3955; A61P 35/00; C07K 7/64; C07K 14/4723

(57) ABSTRACT

Compositions and methods for enhancing immune checkpoint inhibitor therapy of cancer by cotherapy with cyclic peptides are provided. Such cyclic peptides can be naturally occurring or of synthetic origin.

This application is a by-pass continuation application of International Patent Application No. PCT/US2022/080312, filed on Nov. 22, 2022, which claims the benefits of U.S. Provisional Patent Application Ser. No. 63/283,152 filed on Nov. 24, 2021, U.S. Provisional Patent Application Ser. No. 63/283,155 filed on Nov. 24, 2021, and U.S. Provisional Patent Application Ser. No. 63/283,158 filed on Nov. 24, 2021. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

This work was performed with government support under Grant Nos. A122931, AI125141, DE021341 and CA014089 awarded by the National Institutes of Health (NIH). The government has certain rights in the invention.

A computer readable XML file entitled “103388.0015PCT-Sequence-Listing”, that was created on Oct. 27, 2022, and is 29kb in size, contains the sequence listing for this application, has been filed with this application, and is hereby incorporated by reference in its entirety.

### FIELD

The present disclosure is directed to the treatment of cancer, in particular immunotherapeutic interventions.

### BACKGROUND

Immunotherapeutic approaches to treating cancer exploit elements of the patient's own immune system to treat the disease. Such immunotherapies can involve the administration of antibodies, cytokines, and/or cells retrieved from the patient and treated prior to re-administration.

Antibodies utilized in immune checkpoints inhibitor therapy are typically directed to cell surface markers expressed by tumor cells, in order to activate the body's complement system or otherwise identify the cells to the immune system. Alternatively, such antibodies can be directed to cell surface receptors on cancer cells or T cells that result would normally down regulate T-cell activity against cancer cells (e.g., PD-1, PD-L1). Antibodies used for this purpose include Alemtuzumab (a monoclonal antibody directed to CD52, and which activates complement), Atezomlizumab (a monoclonal antibody directed to PD-L1 and which interferes with T-cell deactivation), and Ilipimumab (a monoclonal antibody directed to CTLA4, shifting the T-cell balance towards cytotoxicity). Unfortunately, use of these therapeutic antibodies is associated with unwanted side effects, including precipitation of autoimmune disease, increased rate of infections, and neurological disorders.

Immunotherapies utilizing cells involve removal of cells from the patient, activation and expansion of the cells in culture, and return of the activated cells to the patient. For example, Provenge™ is used to treat prostate cancer, and involves the removal of antigen presenting dendritic cells from the blood by leukapheresis, incubating them with a fusion protein made from elements of GM-CSF and a prostatic acid phosphatase, and reinfusing. The resulting improved presentation of cancer-specific antigens to the immune system is intended to improve the immune response. In another approach, CAR-T inhibitors remove T cells and genetically modifies them to express a chimeric receptor that specifically recognizes target cancer cells. These modified T cells are returned to the patient, where it is hoped that they selectively target the cancer cells. Unfortunately, such approaches are expensive and time consuming, can cause flu-like symptoms, and have produced mixed results.

Thus, there is still a need for a simple and well tolerated immunotherapeutic approach to treating cancer.

### SUMMARY

Cyclic peptides, which can be naturally occurring (e.g., 6-defensins) or synthetic (e.g., analogs of naturally occurring cyclic peptides) have been found to enhance the effects of immune checkpoint inhibitors in the treatment of cancer. Described herein are compositions and methods in which such cyclic peptides are utilized (e.g., at 0.01 mg/kg to 15 mg/kg) in combination with an immune checkpoint inhibitor in order to provide more effective treatment of cancer than is provided by the cyclic peptide or the immune checkpoint inhibitor alone. These and other aspects of the invention disclosed herein will be set forth in greater detail as the patent disclosure proceeds.

Embodiments of the inventive concept include methods of treating cancer by administering an effective amount of a cyclic peptide (e.g., 0.01 mg/kg to 15 mg/kg) and an effective amount of an immune checkpoint inhibitor (ICI, such as antibody directed to PD-1) to a patient in need of treatment for cancer. In some embodiments such a cyclic peptide can be an analog of a θ-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). Such treatment can include or provide induction of tumor (e.g., melanoma) regression, prevention of tumor (e.g., melanoma or colon cancer) reoccurrence, suppression of myeloid-derived suppressor cell infiltration into a tumor microenvironment, and/or augmentation of CD8+ T cell responses in the tumor microenvironment when used in combination with the ICI. In some embodiments use of the cyclic peptides permits a reduction in the dosage of the ICI relative to the amount of ICI administered without the peptide while retaining effectiveness. In some embodiments use of the cyclic peptide reduces or eliminates side effects associated with administration of the ICI (e.g., by permitting a reduction in the amount of ICI needed).

Embodiments of the inventive concept include methods of enhancing immune checkpoint inhibitor therapy by administering an effective amount of a cyclic peptide (0.01 mg/kg to 15 mg/kg) with the immune checkpoint inhibitor (ICI, such as antibody directed to PD-1) to a patient in need of treatment for cancer. In some embodiments such a cyclic peptide can be an analog of a θ-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). In some embodiments the enhancement provided by the cyclic peptide is a reduction in the dosage of the ICI relative to the amount of ICI administered without the peptide while retaining effectiveness. In some embodiments the enhancement provided by the cyclic peptide is reduction or elimination of side effects associated with administration of the ICI (e.g., by permitting a reduction in the amount of ICI needed).

Embodiments of the inventive concept include a pharmaceutical composition that provide a cyclic peptide (e.g., 0.01 mg/kg to 15 mg/kg) in an amount effective to enhance an immune checkpoint therapy. In some embodiments such a cyclic peptide can be an analog of a 0-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). Such a pharmaceutical composition can include an immune checkpoint inhibitor, such as an antibody to PD-1.

Embodiments of the inventive concept include the use of a cyclic peptide in treating cancer in a patient. In such a use an effective amount of a cyclic peptide (e.g., 0.01 mg/kg to 15 mg/kg) is provided in combination with administration of an effective amount of an immune checkpoint inhibitor (ICI, such as an antibody directed to PD-1). In some embodiments such a cyclic peptide can be an analog of a θ-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). Such uses can include induction of tumor (e.g., melanoma) regression, prevention of tumor (e.g., melanoma or colon cancer) reoccurrence, suppression of myeloid-derived suppressor cell infiltration into a tumor microenvironment, augmenting CD8+ T cell responses in the tumor microenvironment when used in combination with the ICI, reducing dosage of the ICI relative to the amount of ICI administered without the peptide while retaining effectiveness, and/or reducing or eliminating side effects associated with administration of the ICI.

Embodiments of the inventive concept include use of a cyclic peptide to enhance immune checkpoint inhibitor therapy, wherein an effective amount of a cyclic peptide (e.g., 0.01 mg/kg to 15 mg/kg) is provided in combination with an immune checkpoint inhibitor (ICI, such as an antibody directed to PD-1). In some embodiments such a cyclic peptide can be an analog of a 0-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). Such enhancements include reducing dosage of the ICI relative to the amount of ICI administered without the peptide while retaining effectiveness and/or reducing or eliminating side effects associated with administration of the ICI.

Embodiments of the inventive concept include a cyclic peptide consisting of peptide 2 (SEQ ID NO. 2) and having the following structure:

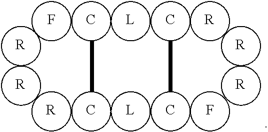

Such embodiments can include a pharmaceutical composition that includes peptide 2 and a pharmaceutically acceptable excipient.

Embodiments of the inventive concept include a kit that includes a cyclic peptide and an immune checkpoint inhibitor (ICI, such as an antibody to PD-1). In some embodiments such a cyclic peptide can be an analog of a θ-defensin. Suitable analogs include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16). In other embodiments such a cyclic peptide can be a naturally occurring θ-defensin, such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33). The cyclic peptide is provided in an amount effective to enhance activity of the immune checkpoint inhibitor (e.g., 0.01 mg/kg to 15 mg/kg). In some embodiments the cyclic peptide and the immune checkpoint inhibitor are comingled. Such a kit can include instructions for use of the cyclic peptide in combination with the immune checkpoint inhibitor to treat cancer.

Various objects, features, aspects and advantages of the inventive subject matter will become more apparent from the following detailed description of preferred embodiments, along with the accompanying drawing figures.

### BRIEF DESCRIPTION OF THE FIGURES

FIG. 1 shows exemplary cyclic peptide 1 (SEQ ID NO. 1), peptide 2 (SEQ ID NO. 2), and peptide 3 (SEQ ID NO. 3).

FIG. 2 shows additional exemplary cyclic peptides peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), and peptide 13 (SEQ ID NO. 13).

FIG. 3 shows a typical protocol for co-administration of a cyclic peptide described herein and an immune checkpoint inhibitor (IC), in this instance anti-PD-1.

FIG. 4 shows cumulative survival curves resulting from the indicated treatments (efficacy for treatment with peptide 1 (SEQ ID NO. 1) and an anti-PD-1 versus saline or anti-PD-1 alone (P<0.005).

FIG. 5 shows the results on tumor growth in mice treated with peptide 1 (SEQ ID NO. 1) in combination with an anti-PD-1 antibody (an IgG2a kappa isotype, clone: RMP1-14, obtained from BioXcell).

FIG. 6 shows a protocol for cotherapy of a cyclic peptide described herein with an anti-PD-1 antibody.

FIG. 7 shows results of monotherapy with peptide 1 (SEQ ID NO. 1), peptide 2 (SEQ ID NO. 2), or peptide 3 (SEQ ID NO. 3), cotherapy with peptide 1 (SEQ ID NO. 1), peptide 2 (SEQ ID NO. 2), or peptide 3 (SEQ ID NO. 3) in combination with an anti-PD-1 monoclonal antibody, and monotherapy with the anti-PD-1 monoclonal antibody in mice that had been challenged with MC38 mouse colon adenocarcinoma cells.

FIG. 8 (upper panel) shows tumors in naïve control mice formed by challenged with MC38 mouse colon adenocarcinoma cells. The lower panel of FIG. 8 shows results from when mice rendered MC38 tumor-free (as in FIG. 7) were rechallenged with MC38 cells and observed for the tumor development for 30 days after rechallenge.

FIG. 9 shows results of treatment with a cyclic peptide (0.1 mg/kg) with 3 doses of anti-PD-1 monoclonal antibody (1 mg/kg), or saline in mice.

FIG. 10 shows results of treatment with peptide 1 (SEQ ID NO. 1), 2, or 3 in combination with anti-PD-1 antibody (Panel A of FIG. 10). Panel B shows the results of the combination therapy on expression of exhaustion marker TIM3 on CD8+ T cells.

FIGS. 11A to 11H show the structures of cyclic peptides peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

FIG. 12 shows a typical protocol for co-administration of a cyclic peptide (in this instance peptide 17 (SEQ ID NO. 17)) and an ICI.

FIG. 13 shows cumulative survival curves resulting from the indicated treatments shown in FIG. 12 (efficacy for treatment with peptide 17 and an anti-PD-1 versus saline or anti-PD-1 alone (P<0.005).

### DESCRIPTION

Synthetic and naturally occurring cyclic peptides and their use in enhancing immune checkpoint inhibitor therapy are described herein. Such synthetic cyclic peptides can be effective at doses ranging from 0.001 mg/kg to 50 mg/kg, 0.005 mg/kg to 30 mg/kg, 0.01 mg/kg to 15 mg/kg, or intermediate ranges. One should appreciate that the disclosed techniques provide many advantageous technical effects including, but not limited to, improving the effectiveness of immunotherapeutic approaches to treating cancer. Suitable cyclic peptides can be produced by known peptide synthesis methods, such as those described in Gunasekera et al., Int J Pept Res Ther (2013) 19:43-54.

Within the context of this application an effective synthetic or naturally occurring cyclic peptide is one that improves the effects of anti-cancer therapy (e.g., reduction in tumor mass or number of cancer cells, inhibition of cancer cell proliferation, reduction of metastasis, increased survival time, prevention of re-occurrence, etc.) with an immune checkpoint inhibitor at concentrations attainable in a human being following administration by oral administration, injection, infusion, inhalation, and/or application to an ocular or mucus membrane. Similarly, methods of the inventive concept can utilize an effective amount of an effective synthetic or naturally occurring cyclic peptide in cotherapy with an immune checkpoint inhibitor in the treatment of cancer. Within the context of this application, an effective amount is an amount of an synthetic or naturally occurring cyclic peptide that is sufficient to improve the effects of immune checkpoint inhibitor therapy in the treatment of cancer. Such effective synthetic or naturally occurring cyclic peptides can be identified using methods as detailed below. Similarly, effective amounts of such effective synthetic or naturally occurring cyclic peptides can be determined using methods as detailed below.

In some embodiments such a cyclic peptides can be a θ-defensin or an analog of a 0-defensin. An analog of a θ-defensin analog can be a cyclic peptide having about 40%, 50%, 60%, 70%, 80%, 90% or greater sequence identity with a naturally occurring θ-defensin peptide sequence. A θ-defensin analog can incorporate one, two, three, or more core features of a naturally occurring θ-defensin. Exemplary core features include cyclic structure, the presence of one, two, three, or more disulfide bonds within the peptide (e.g., between pairs of cysteines of the analog), having a positive charge when in solution under physiological conditions, and the presence of beta pleated sheet secondary structure. Such θ-defensin analogs can include 10, 11, 12, 13, 14, 15, 16, 17, 18, 19, 20, or more than 20 amino acids, and in some embodiments can incorporate non-naturally occurring amino acids. An analog of a θ-defensin can include one or more L-amino acid(s), one or more D-amino acid(s), and/or a mixture of L- and D-amino acids. In some embodiments non-peptide bonds can be utilized between adjacent amino acid residues of a θ-defensin analog. θ-defensin analogs can represent one or more deletion or substitution of amino acids of a naturally occurring θ-defensin sequence. Such substitutions can be conservative (e.g., where the substituted amino acid(s) retain(s) charge, hydrophobicity, hydrophilicity, and/or steric properties of the naturally occurring amino acid). In some embodiments θ-defensin analogs can include grafting or conjugation of non-peptide moieties, for example polyethylene glycol and/or other hydrophilic polymers, cell-receptor targeting moieties, and/or moieties that aid in processing/purification.

The following discussion provides many example embodiments. Although any one embodiment may represent a single combination of elements, the disclosed subject matter is considered to include any or all possible combinations of the disclosed elements. Thus, if one embodiment comprises elements A, B, and C, and a second embodiment comprises elements B and D, then the present disclosure is also considered to include other remaining combinations of A, B, C, or D, even if not explicitly disclosed.

In some embodiments, the numbers expressing quantities of ingredients, properties such as concentration, reaction conditions, and so forth, used to describe and claim certain embodiments are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of an element otherwise claimed. No language in the specification should be construed as indicating any non-claimed element essential to its practice.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

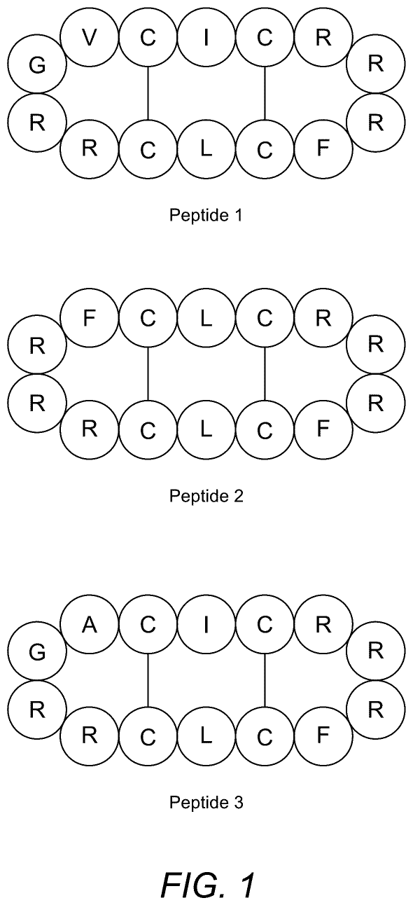

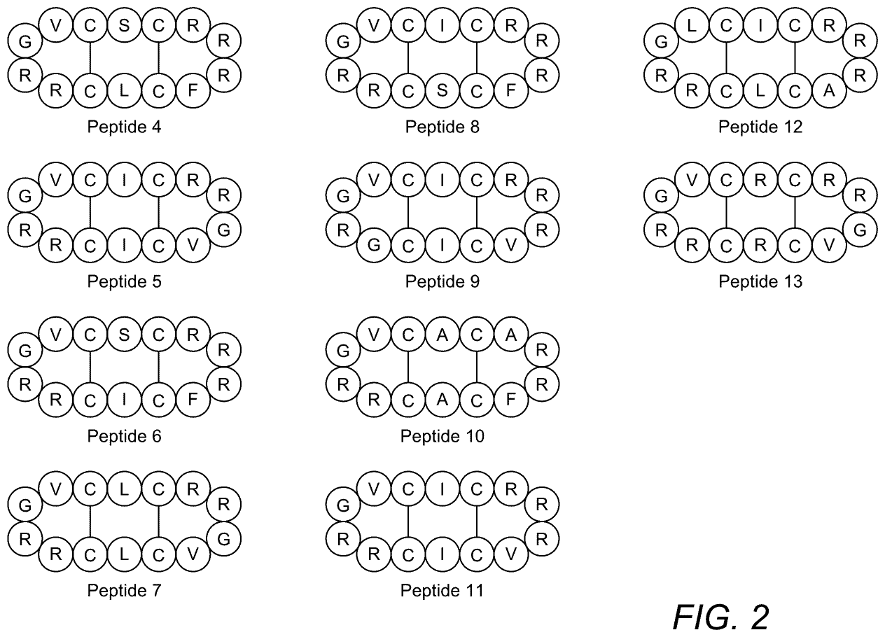

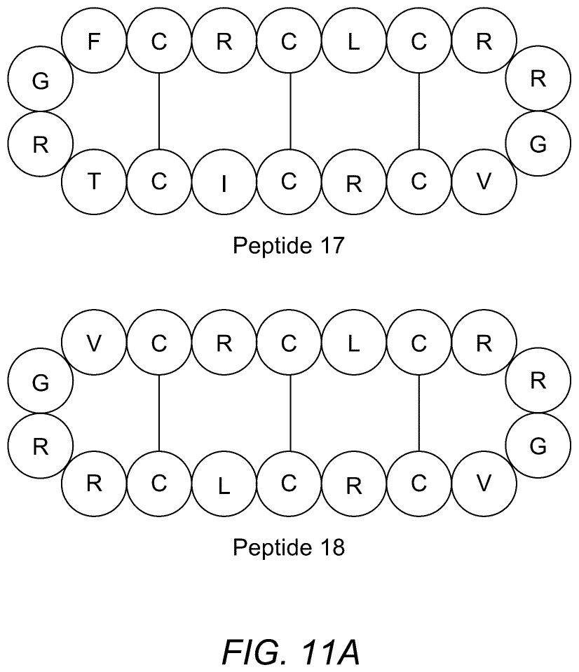

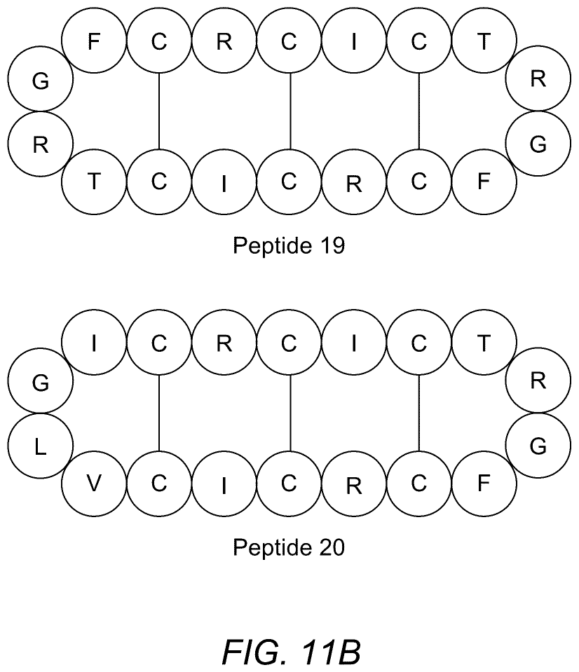

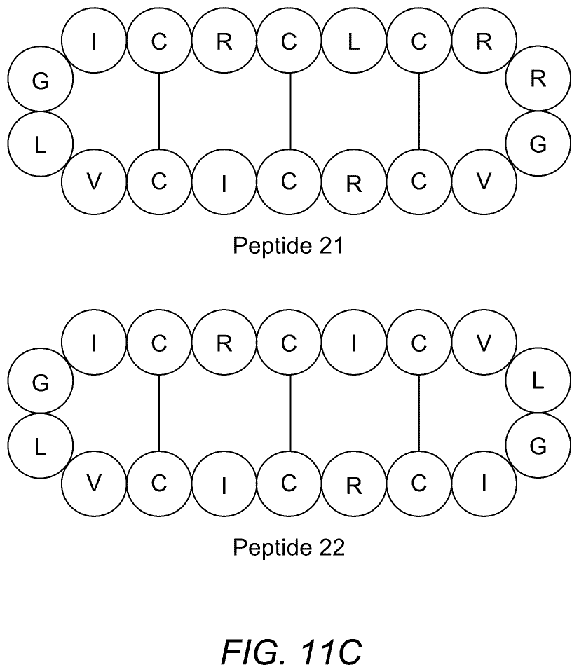

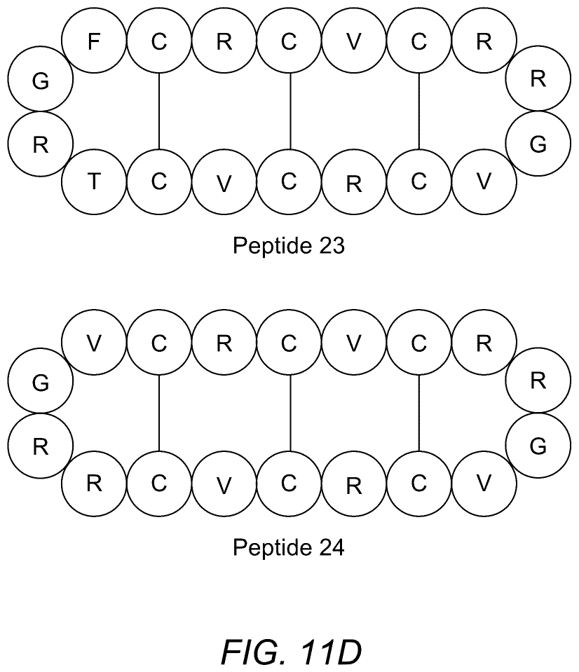

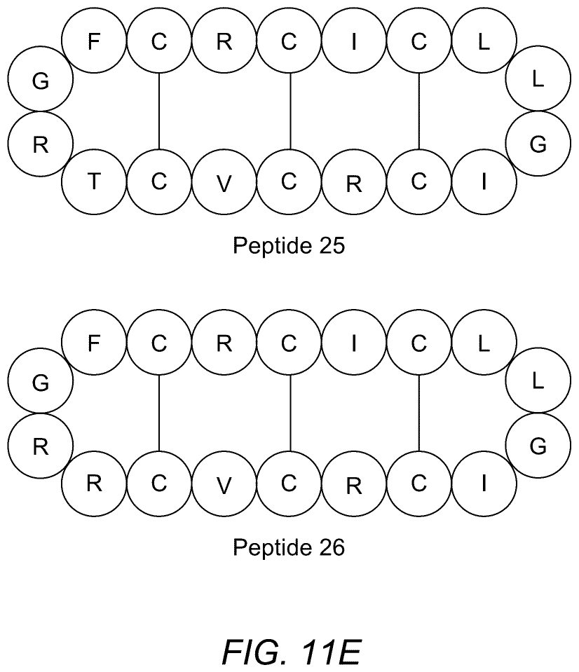

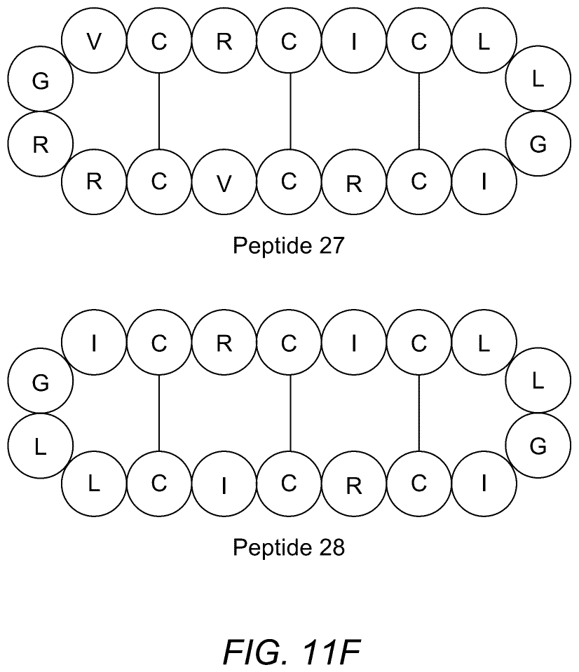

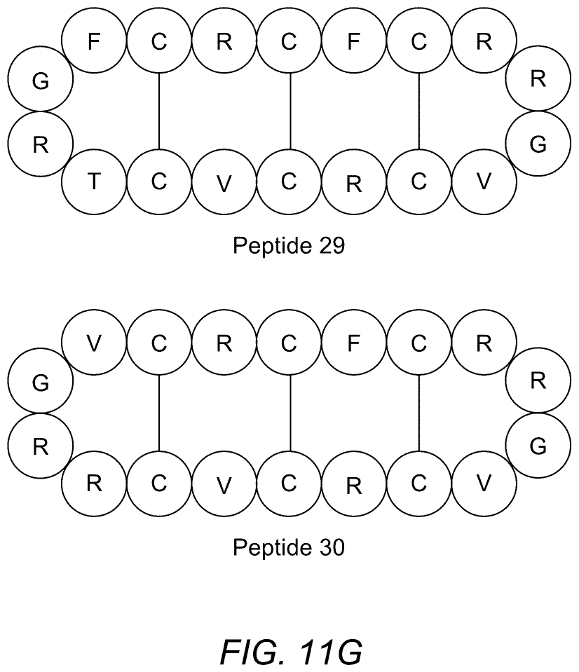

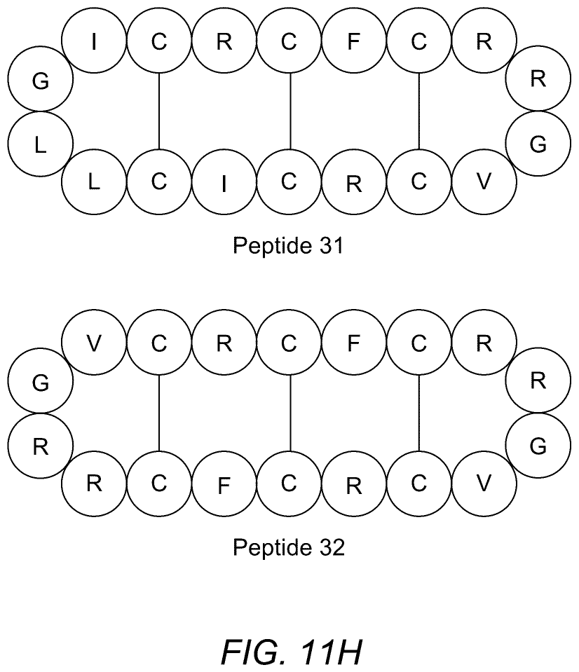

Suitable cyclic peptides have the effect of enhancing an immune checkpoint therapy. Such cyclic peptides include a “CXC box” structure defined by two pairs of cysteines, joined by a pair of disulfide bonds that span the cyclic structure. Otherwise adjacent cysteines within the CXC box are separated by a single hydrophobic amino acid, or in some embodiments by a positively charged amino acid such as arginine. Such peptides also include multiple arginines, and in preferred embodiments at least one arginine triplet consisting of three immediately adjacent arginines. These cyclic peptides have a net positive charge at neutral pH. Examples of structures of suitable synthetic cyclic peptides that are analogs of naturally occurring cyclic peptides are shown in FIG. 1 (peptide 1 (SEQ ID NO. 1), peptide 2 (SEQ ID NO. 2), and peptide 3) and in FIG. 2 (peptides 4 to 13). Structures of suitable naturally occurring cyclic peptides are shown in FIGS. 11A to 11H.

Suitable cyclic peptides that can be used in methods of the inventive concept can be naturally occurring cyclic peptides (e.g., RTD-1) and/or analogs of naturally occurring cyclic peptides. Suitable analogs of naturally occurring cyclic peptides include peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16) (see Table 1). It should be appreciated that while the sequences provided in Table 1 are provided as linear text, the peptides represented are cyclic (i.e., without a free amino and/or carboxyl terminus). Such cyclic peptides are peptide drugs that provide an immunomodulatory activity without immunosuppression.

TABLE 1

| | | Sequence |
|---|---|---|
| Cyclic | Amino Acid | Identification |
| Peptide | Sequence | Number |
| Peptide 1 | CICRRRFCLCRRGV | SEQ ID NO. 1 |
| Peptide 2 | CLCRRRFCLCRRRF | SEQ ID NO. 2 |
| Peptide 3 | CICRRRFCLCRRGA | SEQ ID NO. 3 |
| Peptide 4 | CSCRRRFCLCRRGV | SEQ ID NO. 4 |
| Peptide 5 | CICRRGVCICRRGV | SEQ ID NO. 5 |
| Peptide 6 | CSCRRRFCICRRGV | SEQ ID NO. 6 |
| Peptide 7 | CLCRRGVCLCRRGV | SEQ ID NO. 7 |
| Peptide 8 | CICRRRFCSCRRGV | SEQ ID NO. 8 |
| Peptide 9 | CICRRRVCICGRGV | SEQ ID NO. 9 |
| Peptide 10 | CACARRFCACRRGV | SEQ ID NO. 10 |
| Peptide 11 | CICRRRVCICRRGV | SEQ ID NO. 11 |
| Peptide 12 | CICRRRACLCRRGL | SEQ ID NO. 12 |
| Peptide 13 | CRCRRGVCRCRRGV | SEQ ID NO. 13 |
| Peptide 14 | GFCRCRRGVCRCTR | SEQ ID NO. 14 |
| Peptide 15 | GVCIVRRRFCLCRR | SEQ ID NO. 15 |
| Peptide 16 | GVCLCIRGRCRCRR | SEQ ID NO. 16 |

As noted above, suitable cyclic peptides can be naturally occurring, and isolated from natural sources as described in Garcia et al., Infection and Immunity (2008) 76:5883-5891. Alternatively, such naturally occurring cyclic peptides can be produced by synthetic methods, such as those described in Gunasekera et al., Int J Pept Res Ther (2013) 19:43-54. Examples of suitable naturally occurring cyclic peptides are provided in Table 2. It should be appreciated that while the sequences provided in Table 2 are provided as linear text, the peptides represented are cyclic (i.e., without a free amino and/or carboxyl terminus).

TABLE 2

| | | Sequence |
|---|---|---|
| Cyclic | Amino Acid | Identification |
| Peptide | Sequence | Number |
| Peptide 17 | GFCRCLCRRGVCRCICTR | SEQ ID NO. 17 |
| Peptide 18 | GVCRCLCRRGVCRCLCRR | SEQ ID NO. 18 |
| Peptide 19 | GFCRCICTRGFCRCICTR | SEQ ID NO. 19 |
| Peptide 20 | GICRCICTRGFCRCICVL | SEQ ID NO. 20 |
| Peptide 21 | GICRCLCRRGVCRCICVL | SEQ ID NO. 21 |
| Peptide 22 | GICRCICVLGICRCICVL | SEQ ID NO. 22 |
| Peptide 23 | CVCRRGVCRCVCTRGFCR | SEQ ID NO. 23 |
| Peptide 24 | CVCRRGVCRCVCRRGVCR | SEQ ID NO. 24 |
| Peptide 25 | CICLLGICRCVCTRGFCR | SEQ ID NO. 25 |
| Peptide 26 | CICLLGICRCVCRRGFCR | SEQ ID NO. 26 |
| Peptide 27 | CICLLGICRCVCRRGVCR | SEQ ID NO. 27 |
| Peptide 28 | CICLLGICRCICLLGICR | SEQ ID NO. 28 |
| Peptide 29 | CFCRRGVCRCVCTRGFCR | SEQ ID NO. 29 |
| Peptide 30 | CFCRRGVCRCVCRRGVCR | SEQ ID NO. 30 |
| Peptide 31 | CFCRRGVCRCICLLGICR | SEQ ID NO. 31 |
| Peptide 32 | CFCRRGVCRCFCRRGVCR | SEQ ID NO. 32 |
| Peptide 33 | CFCRRGVCRCFCRRGVCR | SEQ ID NO. 33 |

It is believed that such cyclic peptides can augment ICI therapy by counteracting immunosuppressive pathways in the tumor microenvironment (TME). As shown below, naturally occurring and cyclic peptides can inhibit immunosuppressive cytokines, inhibit infiltration of tumor-promoting myeloid-derived suppressor cells (MDSCs) into the TME, recruit cytotoxic T cells, and reduce T cell exhaustion.

### Immune Checkpoint Inhibitors

As described above, the cyclic peptides are useful in combination with immune checkpoint inhibitors. Immune checkpoint inhibitors include, but are not limited to, anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), or anti-T-cell immunoreceptor with Ig and ITIM domains (TIGIT) agents/inhibitors.

In some embodiments, immune checkpoint inhibitors include, but are not limited to anti-2B4, anti-BTLA, anti-CD160, anti-CTLA-4, anti-LAG-3, anti-PD-1, anti-TIM-3, or anti-TIGIT antibodies. In some embodiments, immune checkpoint inhibitors include, but are not limited to anti-2B4, anti-BTLA, anti-CD160, anti-CTLA-4, anti-LAG-3, anti-TIM-3, or anti-TIGIT antibodies.

Inhibitors to immune checkpoint ligands include, but are not limited to, anti-CD48 antigen (CD48), anti-CD80 antigen (CD80), anti-CD86 antigen (CD86), anti-CD112 antigen (CD112), anti-CD155 antigen (CD155), anti-carcinoembryonic antigen-related cell adhesion molecule 1 (CEACAM1), anti-fibrinogen-like protein 1 (FGL1), anti-galectin-9 (Gal-9), anti-HLA class I histocompatibility antigen, alpha chain B (HLA-B), anti-HLA class I histocompatibility antigen, alpha chain C (HLA-C), anti-HLA class I histocompatibility antigen, alpha chain E (HLA-E), anti-HLA class I histocompatibility antigen, alpha chain G (HLA-G), anti-high mobility group protein B1 (HMG1), anti-herpesvirus entry mediator A (HVEM), anti-programmed cell death 1 ligand 1 (PD-L1), and anti-programmed cell death 1 ligand 2 (PD-L2) agents/inhibitors. In some embodiments, a cyclic peptide as described herein, is administered in combination with an inhibitor to an immune checkpoint ligand other than a PD-L1/2 ligand. In some embodiments, inhibitors to immune checkpoint ligands include, but are not limited to anti-CD48, anti-CD80, anti-CD86, anti-CD112, anti-CD155, anti-CEACAM1, anti-FGL1, anti-Gal-9, anti-HLA-B, anti-HLA-C, anti-HLA-E, anti-HLA-G, anti-HMG1, anti-HVEM, anti-PD-L1, and anti-PD-L2 antibodies. In some embodiments, inhibitors to immune checkpoint ligands include, but are not limited to anti-CD48, anti-CD80, anti-CD86, anti-CD112, anti-CD155, anti-CEACAM1, anti-FGL1, anti-Gal-9, anti-HLA-B, anti-HLA-C, anti-HLA-E, anti-HLA-G, anti-HMG1, and anti-HVEM.

### Anti-2B4/Anti-CD48 Agents

As used herein, “2B4” refers to the natural killer cell (2B4) receptor. Other names include NK cell activation-ligand (NAIL), NK cell type I receptor protein 2B4, signaling lymphocytic activation molecule 4 (SLAM4), and CD244 (cluster of differentiation 244). 2B4 has at least one ligand, CD48 (cluster of differentiation 48). In some embodiments, targeting 2B4 restores immune function in the tumor microenvironment. In some embodiments, the anti-2B4 or anti-CD48 agent is an antibody, a peptide, a small molecule or a nucleic acid.

In some embodiments, the anti-2B4 agent is an anti-2B4 antibody. In some embodiments, the anti-CD48 agent is an anti-CD48 antibody.

“Anti-2B4 antibody” refers to an antibody directed towards the natural killer cell (2B4) receptor. In some embodiments, an anti-2B4 antibody binds an epitope of 2B4 which blocks the binding of 2B4 to any one or more of its putative ligands. In some embodiments, an anti-2B4 antibody binds an epitope of a 2B4 protein which blocks the binding of 2B4 to CD48. “Anti-CD48 antibody” refers to an antibody directed towards CD48 antigen (CD48).

The terms “antibody” and “antibodies” as used herein is inclusive of all types of immunoglobulins, including IgG, IgM, IgA, IgD, and IgE, or active fragments thereof, that may be appropriate for the medical uses disclosed herein. The antibodies may be monoclonal or polyclonal and may be of any species of origin, including, for example, mouse, rat, rabbit, horse, or human. Antibody fragments that retain specific binding to the protein or epitope, for example, 2B4 or CD48, bound by the antibody used in the present disclosure are included within the scope of the term “antibody.” The antibodies may be chimeric or humanized, particularly when they are used for therapeutic purposes. Antibodies and antibody fragments may be obtained or prepared using various methods.

### Anti-BTLA/Anti-HVEM Agents

As used herein, “BTLA” refers to B- and T-lymphocyte attenuator (BTLA) receptor. Other names include B- and T-lymphocyte-associated protein and CD272 (cluster of differentiation 272). BTLA has at least one ligand, herpesvirus entry mediator A (HVEM). In some embodiments, targeting BTLA restores immune function in the tumor microenvironment.

In some embodiments, the anti-BTLA or anti-HVEM agent is an antibody, a peptide, a small molecule or a nucleic acid.

“Anti-BTLA antibody” refers to an antibody directed towards the B- and T-lymphocyte attenuator (BTLA) receptor. In some embodiments, an anti-BTLA antibody binds an epitope of BTLA which blocks the binding of BTLA to any one or more of its putative ligands. In some embodiments, an anti-BTLA antibody binds an epitope of a BTLA protein which blocks the binding of BTLA to HVEM. “Anti-HVEM antibody” refers to an antibody directed towards herpesvirus entry mediator A (HVEM).

### Anti-CD160/Anti-MHC Class I Agents

As used herein, “CD160” refers to CD160 antigen (CD160) receptor. Other names include natural killer cell receptor BY55 (BY55) and CD160 (cluster of differentiation 160). CD160 has at least two ligands, herpesvirus entry mediator A (HVEM) and major histocompatibility complex (MHC) class I proteins including HLA class I histocompatibility antigen, alpha chain B (HLA-B), HLA class I histocompatibility antigen, alpha chain C (HLA-C), HLA class I histocompatibility antigen, alpha chain E (HLA-E), and HLA class I histocompatibility antigen, alpha chain G (HLA-G). In some embodiments, targeting CD160 restores immune function in the tumor microenvironment.

In some embodiments, the anti-CD160, anti-HLA-B, anti-HLA-C, anti-HLA-E, anti-HLA-G, or anti-HVEM agent is an antibody, a peptide, a small molecule or a nucleic acid.

In some embodiments, the anti-CD160 agent is an anti-CD160 antibody. In some embodiments, the anti-HLA-B agent is an anti-HLA-B antibody. In some embodiments, the anti-HLA-C agent is an anti-HLA-C antibody. In some embodiments, the anti-HLA-E agent is an anti-HLA-E antibody. In some embodiments, the anti-HLA-G agent is an anti-HLA-G antibody. In some embodiments, the anti-HVEM agent is an anti-HVEM antibody.

“Anti-CD160 antibody” refers to an antibody directed towards the CD160 antigen (CD160) receptor. In some embodiments, an anti-CD160 antibody binds an epitope of CD160 which blocks the binding of CD160 to any one or more of its putative ligands. In some embodiments, an anti-CD160 antibody binds an epitope of a CD160 protein which blocks the binding of CD160 to HLA-B, HLA-C, HLA-E, HLA-G, or HVEM. “Anti-HLA-B antibody” refers to an antibody directed towards HLA class I histocompatibility antigen, alpha chain B (HLA-B). “Anti-HLA-C antibody” refers to an antibody directed towards HLA class I histocompatibility antigen, alpha chain C (HLA-C). “Anti-HLA-E antibody” refers to an antibody directed towards HLA class I histocompatibility antigen, alpha chain E (HLA-E). “Anti-HLA-G antibody” refers to an antibody directed towards HLA class I histocompatibility antigen, alpha chain G (HLA-G).

### Anti-CTLA-4/Anti-CD80/Anti-CD86 Agents

As used herein, “CTLA-4” or “CTLA4” refers to cytotoxic T-lymphocyte protein 4 (CTLA-4) receptor. Other names include cytotoxic T-lymphocyte-associated antigen 4 (CTLA-4) and CD152 (cluster of differentiation 152). CTLA-4 has at least two ligands, T-lymphocyte activation antigen CD80 (CD80) and T-lymphocyte activation antigen CD86 (CD86). In some embodiments, targeting CTLA-4 restores immune function in the tumor microenvironment.

In some embodiments, the anti-CTLA-4, anti-CD80, or anti-CD86 agent is an antibody, a peptide, a small molecule or a nucleic acid.

In some embodiments, the anti-CTLA-4 agent is an anti-CTLA-4 antibody. In some embodiments, the anti-CD80 agent is an anti-CD80 antibody. In some embodiments, the anti-CD86 agent is an anti-CD86 antibody. Anti-CTLA-4 antibody” refers to an antibody directed towards the cytotoxic T-lymphocyte protein 4 (CTLA-4) receptor. In some embodiments, an anti-CTLA-4 antibody binds an epitope of CTLA-4 which blocks the binding of CTLA-4 to any one or more of its putative ligands. In some embodiments, an anti-CTLA-4 antibody binds an epitope of a CTLA-4 protein which blocks the binding of CTLA-4 to CD80 or CD86. “Anti-CD80 antibody” refers to an antibody directed towards CD80 antigen (CD80). “Anti-CD86 antibody” refers to an antibody directed towards CD86 antigen (CD80).

### Anti-LAG-3/Anti-FGL1/Anti-MHC Class II Agents

As used herein, “LAG-3” refers to lymphocyte activation gene 3 protein (LAG-3) receptor. Other names include CD223 (cluster of differentiation 223). LAG-3 has at least two ligands, fibrinogen-like protein 1 (FGL1) and major histocompatibility complex (MHC) class II proteins. In some embodiments, targeting CD160 restores immune function in the tumor microenvironment.

In some embodiments, the anti-LAG-3, anti-FGL1, or anti-MHC class II agent is an antibody, a peptide, a small molecule or a nucleic acid.

In some embodiments, the anti-LAG-3 agent is an anti-LAG-3 antibody. In some embodiments, the anti-FGL1 agent is an anti-FGL1 antibody. In some embodiments, the anti-MHC class II agent is an anti-MHC class II antibody.

“Anti-LAG-3 antibody” refers to an antibody directed towards the lymphocyte activation gene 3 protein (LAG-3) receptor. In some embodiments, an anti-LAG-3 antibody binds an epitope of LAG-3 which blocks the binding of LAG-3 to any one or more of its putative ligands. In some embodiments, an anti-LAG-3 antibody binds an epitope of a LAG-3 protein which blocks the binding of LAG-3 to FGL1, or MHC class II. “Anti-FGL1 antibody” refers to an antibody directed towards fibrinogen-like protein 1 (FGL1). “Anti-MHC class II antibody” refers to an antibody directed towards major histocompatibility complex (MHC) class II protein.

### Anti-PD-1/Anti-PD-L1/Anti-PDL2 Agents

As used herein, “PD-1” or “PD1” refers to the Programmed Death 1 (PD-1) receptor. Other names include programmed cell death protein 1 and CD279 (cluster of differentiation 279). PD-1 has two ligands, PD-L1 and PD-L2. In some embodiments, targeting PD-1 restores immune function in the tumor microenvironment. As used herein, “PD-L1” or “PDL1” refers to the programmed death ligand 1 (PD-L1). As used herein, “PD-L2” or “PDL2” refers to the programmed death ligand 2 (PD-L2). In some embodiments, the anti-PD-1 or anti-PDL-1 agent is an antibody, a peptide, a small molecule or a nucleic acid.

In some embodiments, the anti PD-1 agent for use in combination with compound described herein is nivolumab, pembrolizumab, atezolizumab, durvalumab, pidilizumab, avelumab, TSR-042, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CSIOOI, Sym-021, SHR-1316, PF-06801591, LZM009, KN-035, AB122, genolimzumab (CBT-501), FAZ-053, CK-301, AK 104, or GLS-010, BGB-108, SHR-1210, PDR-001, PF-06801591, STI-1110, mDX-400, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, or AMP-514 (MEDI0680).

In some embodiments, the anti PD-1 agent is an anti PD-1 antibody. “Anti-PD-1 antibody” refers to an antibody directed towards programmed death protein 1 (PD1). In some embodiments, an anti-PD-1 antibody binds an epitope of PD-1 which blocks the binding of PD-1 to any one or more of its putative ligands. In some embodiments, an anti-PD1 antibody binds an epitope of a PD-1 protein which blocks the binding of PD-1 to PD-L1 and/or PD-L2.

Exemplary anti-PD-1 antibodies include but are not limited to: nivolumab/MDX-1106/BMS-9300/ONO1152, a fully human IgG4 anti-PD-1 monoclonal antibody; pidilizumab (MDV9300/CT-011), a humanized IgG1 monoclonal antibody; pembrolizumab (MK-3475/pembrolizumab/lambrolizumab), a humanized monoclonal IgG4 antibody; durvalumab (MEDI-4736) and atezolizumab.

In some embodiments, the anti-PD-1 antibody is nivolumab (OPDIVO®, Bristol-Myers Squibb), pembrolizumab (KEYTRUDA®, Merck), cemiplimab (Libtayo), labrolizumab (Merck), or BGB-A317.

In some embodiments, the anti-PD1 antibody is an antibody set forth in U.S. Pat. Nos. 7,029,674, 7,488,802, 7,521,051, 8,008,449, 8,354,509, 8,617,546, 8,709,417, or WO2014/179664.

In some embodiments, the anti PD-1 agent for use in combination is atezolizumab, avelumab, AMP-224, MEDI-0680, RG-7446, GX-P2, durvalumab, KY-1003, KD-033, MSB-0010718C, TSR-042, ALN-PDL, STI-A1014, CX-072, BMS-936559, KN035, CK-301 (Checkpoint Therapeutics), AUNP12, CA-170 (Aurigene/Curis), MEDI4736, MSB0010718C, MDX 1105-01, or BMS-986189.

In some embodiments, the anti PD-L1 agent is an anti PD-L1 antibody. “Anti-PD-L1 antibody” refers to an antibody directed towards programmed death ligand 1 (PD-L1). Anti-PD-L1 antibodies include: avelumab; BMS-936559, a fully human IgG4 antibody; atezolizumab (MPDL3280A/RG-7446), a human monoclonal antibody; MEDI4736; MSB0010718C, and MDX 1105-01.

In some embodiments, the anti-PD-L1 antibody is avelumab (Bavencio®, Merck KGA/Pfizer), durvalumab (AstraZeneca) and atezolizumab (TECENTRIQ®, Roche).

Additional exemplary antibodies include, but are not limited to, the antibodies set forth in U.S. Pat. Nos. 8,217,149, 8,383,796, 8,552,154 and 8,617,546.

Peptide anti-PD-1/PD-L1 agents include AUNP12 (a 29-mer peptide by Aurigene and Laboratoires Pierre Fabre), CA-170 (Aurigene/Curis), BMS-986189 (a macrocyclic peptide by BMS).

Small molecule anti-PD-1/PD-L1 agents include those described in WO/2020/086556, WO/2020/014643, WO/2019/204609, WO/2019/160882, WO/2018/195321, WO2018026971, US20180044329, US20180044305, US20180044304, US20180044303, US20180044350, US20180057455, US20180057486, US20180045142, WO20180044963, WO2018044783, WO2018009505, WO20180044329, WO2017066227, WO2017087777, US20170145025, WO2017079669, WO2017070089, US2017107216, WO2017222976, US20170262253, WO2017205464, US20170320875, WO2017192961, WO2017112730, US20170174679, WO2017106634, WO2017202744, WO2017202275, WO2017202273, WO2017202274, WO2017202276, WO2017180769, WO2017118762, WO2016041511, WO2016039749, WO2016142835, WO2016142852, WO2016142886, WO2016142894, and WO2016142833.

### Anti-TIM-3/Anti-Gal-9/Anti-HMG1/Anti-CEACAM1 Agents

As used herein, “TIM-3” refers to T-cell immunoglobulin mucin receptor 3 (TIM-3) receptor. Other names include hepatitis A virus cellular receptor 2 (HAVcr-2), T-cell immunoglobulin and mucin domain-containing protein 3 (TIMD-3), T-cell membrane protein 3, and CD366 (cluster of differentiation 366). TIM-3 has at least three ligands, galectin-9 (Gal-9), high mobility group protein B1 (HMG1), and carcinoembryonic antigen-related cell adhesion molecule 1 (CEACAM1). In some embodiments, targeting TIM-3 restores immune function in the tumor microenvironment.

In some embodiments, the anti-TIM-3, anti-Gal-9, anti-HMG1, or anti-CEACAM1 agent is an antibody, a peptide, a small molecule or a nucleic acid. In some embodiments, the anti-TIM-3 agent is an anti-TIM-3 antibody. In some embodiments, the anti-Gal-9 agent is an anti-Gal-9 antibody. In some embodiments, the anti-HMG1 agent is an anti-HMG1 antibody. In some embodiments, the anti-CEACAM1 agent is an anti-CEACAM1 antibody.

“Anti-TIM-3 antibody” refers to an antibody directed towards the T-cell immunoglobulin mucin receptor 3 (TIM-3) receptor. In some embodiments, an anti-TIM-3 antibody binds an epitope of TIM-3 which blocks the binding of TIM-3 to any one or more of its putative ligands. In some embodiments, an anti-TIM-3 antibody binds an epitope of a TIM-3 protein which blocks the binding of TIM-3 to Gal-9, HMG1, or CEACAM1. “Anti-Gal-9 antibody” refers to an antibody directed towards galectin 9 (Gal-9). “Anti-HMG1 antibody” refers to an antibody directed towards high mobility group protein B1 (HMG1). “Anti-CEACAM1 antibody” refers to an antibody directed towards carcinoembryonic antigen-related cell adhesion molecule 1 (CEACAM1).

### Anti-TIGIT/Anti-CD112/Anti-CD155 Agents

As used herein, “TIGIT” refers to T-cell immunoreceptor with Ig and ITIM domains (TIGIT) receptor. Other names include V-set and immunoglobulin domain-containing protein 9 (VSIG9) and V-set and transmembrane domain-containing protein 3 (VSTM3). TIGIT has at least two ligands, CD112 (cluster of differentiation 112) and CD155 (cluster of differentiation 155). In some embodiments, targeting TIGIT restores immune function in the tumor microenvironment.

In some embodiments, the anti-TIGIT, anti-CD112, or CD155 agent is an antibody, a peptide, a small molecule or a nucleic acid. In some embodiments, the anti-TIGIT agent is an anti-TIGIT antibody. In some embodiments, the anti-CD112 agent is an anti-CD112 antibody. In some embodiments, the anti-CD155 agent is an anti-CD155 antibody. “Anti-TIGIT antibody” refers to an antibody directed towards the T-cell immunoreceptor with Ig and ITIM domains (TIGIT) receptor. In some embodiments, an anti-TIGIT antibody binds an epitope of TIGIT which blocks the binding of TIGIT to any one or more of its putative ligands. In some embodiments, an anti-TIGIT antibody binds an epitope of a TIGIT protein which blocks the binding of TIGIT to CD112 or CD155. “Anti-CD112 antibody” refers to an antibody directed towards CD112 (cluster of differentiation 112). “Anti-CD155 antibody” refers to an antibody directed towards CD155 (cluster of differentiation 155).

### EXAMPLES

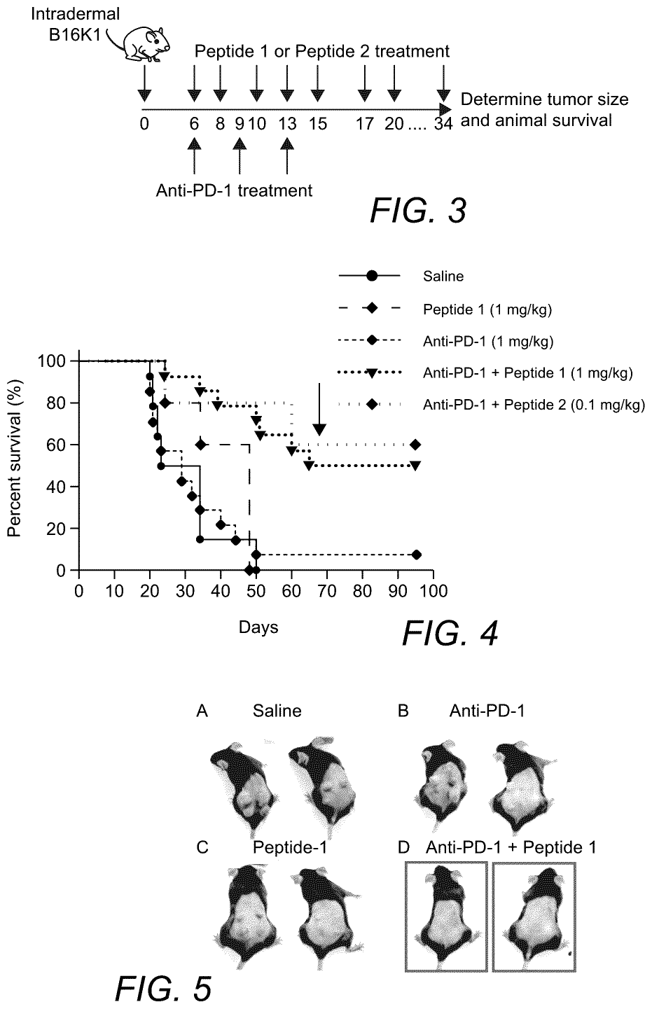

It has been found that ICI therapy directed to PD-1 expression can be augmented using cyclic peptides, including naturally occurring cyclic peptides and analogs of naturally occurring cyclic peptides. A typical protocol useful for identifying or characterizing effective analogs of naturally occurring cyclic peptides or naturally occurring cyclic peptides is shown in FIG. 3.
As shown in FIG. 3, C57BL/6 wild-type (WT) mice were intradermally grafted with 3×10⁵ B16K1 melanoma cells, followed by intraperitoneal (IP) administration of the indicated cyclic peptides (3 times per week for 3 weeks), with or without administration of an anti-PD-1 monoclonal antibody (anti-PD-1 RMP1-14 from BioXcell, 1 mg kg⁻¹) at days 6, 9, and 13 (n=10 mice per group). Other suitable anti-PD-1 antibodies include, but are not limited to, MEDI0680, nivolumab, pembrolizumab, cemiplimab, atezolizumab, dostarlimab, durvalumab, and avelumab. Results for analogs of naturally occurring cyclic peptides Peptide 1 (SEQ ID NO. 1) and Peptide 2 (SEQ ID NO. 2) using a protocol as shown in FIG. 3 are shown in FIG. 4.

FIG. 4 shows cumulative survival curves resulting from the indicated treatments (efficacy for peptide treatment with anti-PD-1 versus saline or anti-PD-1 alone P<0.005). As shown, cotherapy with anti-PD-1 and cyclic peptides enhances anti-PD-1 therapy, and in fact provides an effect that exceeds the summation of effects provided by anti-PD1 or the cyclic peptides as monotherapies. It should also be appreciated that surviving mice were re-challenged with B16K1 cells on day 65 (arrow). Surprisingly, none of these mice developed tumors.

The effect of cotherapy was also studied with anti-PD-1 therapy and cyclic peptides on regression of melanoma. C57BL/6 mice were implanted with B16K1 melanoma and treated as shown in FIG. 3 with saline, anti-PD-1 alone, peptide 1 (SEQ ID NO. 1) alone, or peptide 1 (SEQ ID NO. 1) in combination with anti-PD-1. Typical results are shown in FIG. 5.

Surprisingly, complete tumor regression was obtained in 40% of the mice treated with peptide 1 (SEQ ID NO. 1) in combination with anti-PD-1 antibody compared to 10% of the mice treated with anti-PD-1 alone. This effect was not observed in mice treated with saline or cyclic peptide alone, indicating that cotherapy with cyclic peptide enhances anti-PD-1 therapy. In fact, such cotherapy provides an effect that exceeds the summation of effects provided by anti-PD-1 or the cyclic peptide as monotherapies.

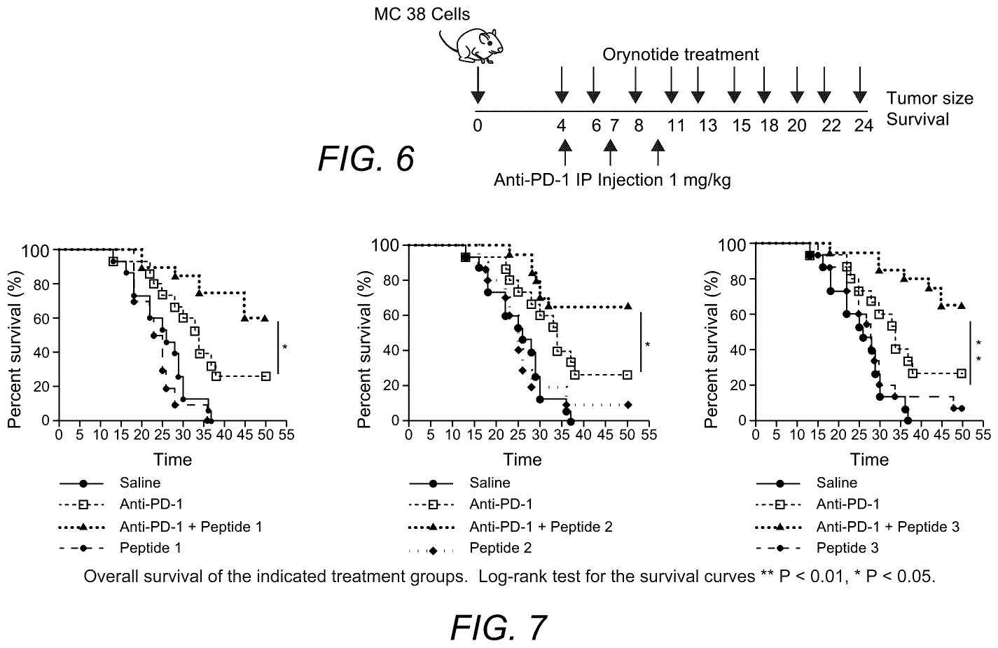

Enhancement was also found of the efficacy of anti-PD-1 therapy in a mouse colon cancer model by coadministration of a cyclic peptide as described herein. A typical protocol for identifying or characterizing effective analogs of naturally occurring cyclic peptides or effective naturally occurring cyclic peptides is shown in FIG. 6.

Mice were challenged with MC38 mouse colon adenocarcinoma cells and treated with peptide 1 (SEQ ID NO. 1), peptide 2 (SEQ ID NO. 2), or peptide 3 (SEQ ID NO. 3) alone, peptide 1 (SEQ ID NO. 1), peptide 2 (SEQ ID NO. 2), or peptide 3 (SEQ ID NO. 3) in combination with an anti-PD-1 monoclonal antibody, or with the anti-PD-1 monoclonal antibody alone. Results are shown in FIG. 7.

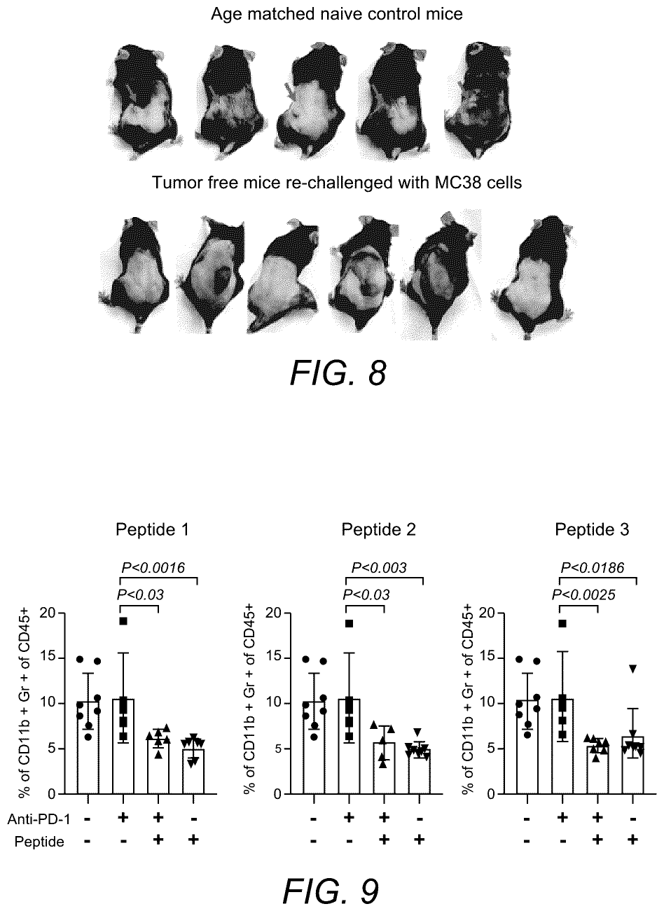

As shown, peptide 1 (SEQ ID NO. 1), peptide 2 (SEQ ID NO. 2), and peptide 3 (SEQ ID NO. 3) all potentiated the effect of the anti-PD-1 monoclonal antibody in regard to survival. Such combination therapy provided an effect that exceeds the summation of effects provided by anti-PD1 or the cyclic peptide as monotherapies. As noted above, rechallenge studies with melanoma indicated that combination therapy with the synthetic cyclic peptide and ICI was effective at preventing reoccurrence on re-introduction of cancer cells. Use of cyclic peptides in combination anti-PD-1 ICI also effectively immunized mice to rechallenge with MC38 colon cancer cells. Results of these studies are shown in FIG. 8. As shown in the upper panel of FIG. 8, MC38 cells formed tumors in naïve control mice. The lower panel of FIG. 8 shows results from when mice rendered MC38 tumor-free (as in FIG. 7) were rechallenged with MC38 cells and observed for the tumor development for 30 days after rechallenge. No tumors formed, demonstrating that tumor immunity provided by combination a cyclic peptide and ICI therapy is likely widely applicable.

It was also found that cyclic peptides can suppress myeloid-derived suppressor cell (MDSC) infiltration into the tumor microenvironment. C57BL/6 mice were implanted subcutaneously with 5×10⁶ MC38 colon adenocarcinoma cells, and subsequently treated with 4 doses of the indicated cyclic peptide (0.1 mg/kg), 4 doses of the indicated a cyclic peptide with 3 doses of an anti-PD-1 monoclonal antibody (1 mg/kg), or saline (control). Tumors were harvested at day 13 and the percent of myeloid-derived suppressor cells (MDSCs) was quantified by FACS.

As shown, peptide 1 (SEQ ID NO. 1), peptide 2 (SEQ ID NO. 2), and peptide 3 (SEQ ID NO. 3) each showed significant activity in reducing the percentage of MDSCs within the tumor microenvironment. While the anti-PD-1 antibody alone had little effect, an enhancement is evident when peptide 1 (SEQ ID NO. 1) is used in combination with the anti-PD-1 monoclonal antibody. This enhanced effect exceeded the sum of the effects of anti-PD-1 monoclonal monotherapy and peptide 1 (SEQ ID NO. 1) monotherapy.

It is believed that cyclic peptides (e.g., peptide 1 (SEQ ID NO. 1), peptide 2 (SEQ ID NO. 2), peptide 3) can provide a less toxic and potentially more effective treatment in a broad range of oncologic indications in which suppression of MDSCs is a treatment goal.

It was also found that the use of cyclic peptides in combination with ICI (e.g., an anti-PD-1 monoclonal antibody) can enhance infiltration of cytotoxic T cells into the tumor microenvironment. C57BL/6 mice were implanted subcutaneously with 5×10⁶ MC38 colon adenocarcinoma cells. Mice were treated with 4 doses of the indicated cyclic peptide (0.1 mg/kg), 4 doses of the indicated cyclic peptide (0.1 mg/kg) with 3 doses of anti-PD-1 monoclonal antibody (1 mg/kg), or saline (control). Tumors were harvested at day 13 and percent of CD8+ T cells was quantified by FACS. Results are shown in FIG. 9.

As shown, each combination of cyclic peptide with anti-PD-1 monoclonal antibody increased CD8+ T cells, reaching statistical significance (Mann-Whitney test) with peptide 1. The effects of such combination therapy exceeded the effect of anti-PD-1 monoclonal antibody monotherapy and monotherapies with the respective cyclic peptides.

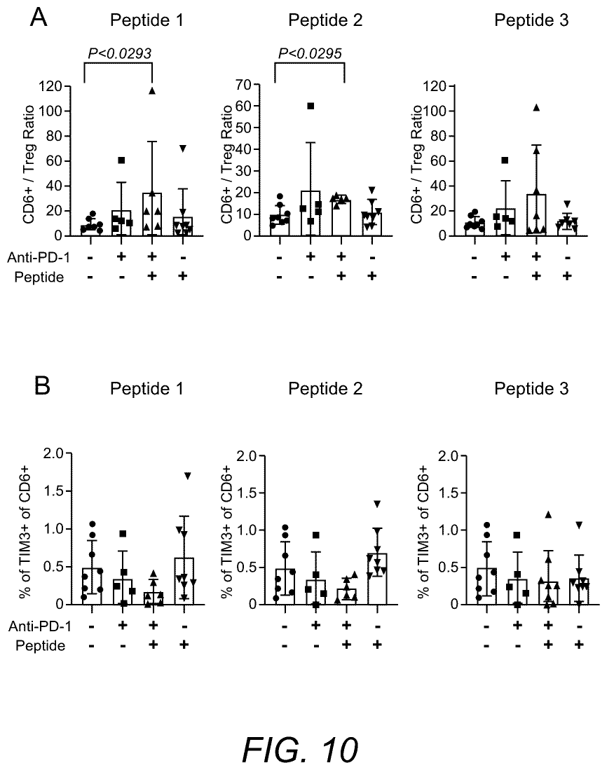

It was also found that cyclic peptides used in combination with an ICI (such as an anti-PD-1 monoclonal antibody) can augment CD8+ T cell responses in the tumor microenvironment (TME). Cell populations from MC38 colon adenocarcinoma tumors were analyzed by FACS following treatment with cyclic peptides in combination with anti-PD-1 (Panel A of FIG. 10) were found to have an increased ratio of CD8+ T cells to T regulatory cells in TME, with peptide 1 (SEQ ID NO. 1) and peptide 2 (SEQ ID NO. 2) effects reaching statistical significance (Mann-Whitney test). Combination therapy also reduced the expression of exhaustion marker TIM3 on CD8+ T cells (Panel B of FIG. 10).

It is believed that modulation of T cell activity in the tumor microenvironment is an important component of the beneficial effect of treatment with a synthetic cyclic peptide, with or without concomitant use of immune checkpoint therapy.

In view of the effects of cyclic peptides (e.g., peptide 1 (SEQ ID NO. 1), peptide 2 (SEQ ID NO. 2), peptide 3) to potentiate immune checkpoint inhibitor (ICI) therapy, it is believed that members of this family of peptide drugs can render ICI efficacious in patients that are currently not successfully treated with current ICI therapy. Similarly, it is believed that such combination therapy can produce remissions of longer duration than are possible with ICI treatment in the absence of synthetic cyclic peptides, and that such combination therapy can enable new remission in patients whose disease initially responded but then progressed on ICI therapy.

In addition, it is believed that cotherapy with cyclic peptides can enable the use of ICI therapy in some first-line indications where ICI alone is not currently feasible. Similarly, the low to nonexistent toxicity of cyclic peptides at effective doses indicates their potential for enabling dose reduction of ICIs (while maintaining efficacy). It is contemplated ICI cyclic peptide combination therapy can be useful as an adjuvant therapy in some oncologic indications.

As shown, use of cyclic peptides can significantly potentiate a broad range of the effects of immune checkpoint therapy. Accordingly, it is believed that cotherapy with one or more cyclic peptides (e.g., peptide 1 (SEQ ID NO. 1), peptide 2 (SEQ ID NO. 2), peptide 3) can permit the use of ICI at lower doses that are currently in use. Such lower doses can reduce the occurrence and/or severity of side effects or adverse immune effects associated with ICI therapy.

Cyclic peptides (e.g., peptide 1 (SEQ ID NO. 1), peptide 2 (SEQ ID NO. 2), peptide 3) may have the effect of down-regulating pro-inflammatory cytokines, such as TNFα and IL-6. It is believed that cyclic peptides can provide a lower toxicity option for down-regulation of TNF-alpha and IL-6 in oncologic indications.

As noted above, suitable cyclic peptides can be naturally occurring cyclic peptides. Suitable naturally occurring cyclic peptides include θ-defensins. It is believed that such naturally occurring cyclic peptides can augment ICI therapy by counteracting immunosuppressive pathways in the tumor microenvironment (TME). Studies with θ-defensins and synthetic analogs of θ-defensins indicate that such cyclic peptides can inhibit immunosuppressive cytokines, inhibit infiltration of tumor-promoting myeloid-derived suppressor cells (MDSCs) into the TME, recruit cytotoxic T cells, and reduce T cell exhaustion.

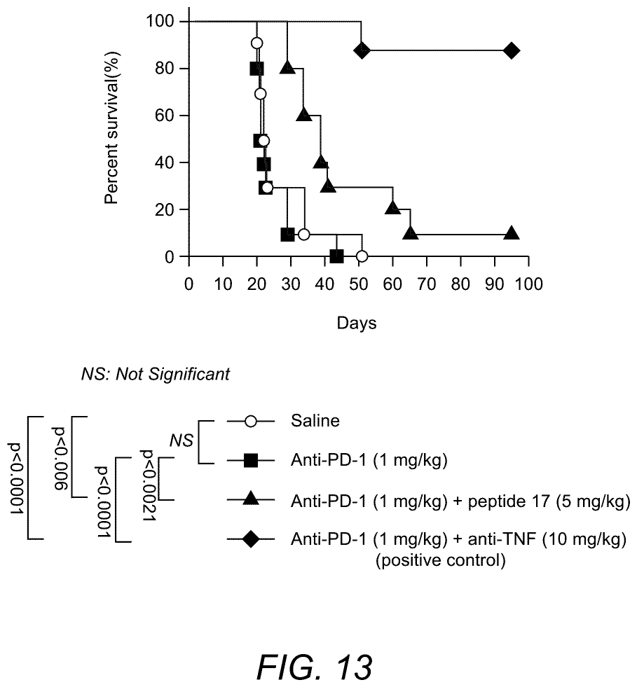

It has been found that ICI therapy directed to PD-1 expression can be augmented using naturally occurring cyclic octadecapeptides (such as peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and/or peptide 33 (SEQ ID NO. 33)), which are shown in FIGS. 11A-H. A typical protocol for identifying an effective naturally occurring cyclic peptide (i.e., one that is effective in enhancing the effects of immune checkpoint therapy) or analog of a naturally occurring cyclic peptide is shown in FIG. 13.

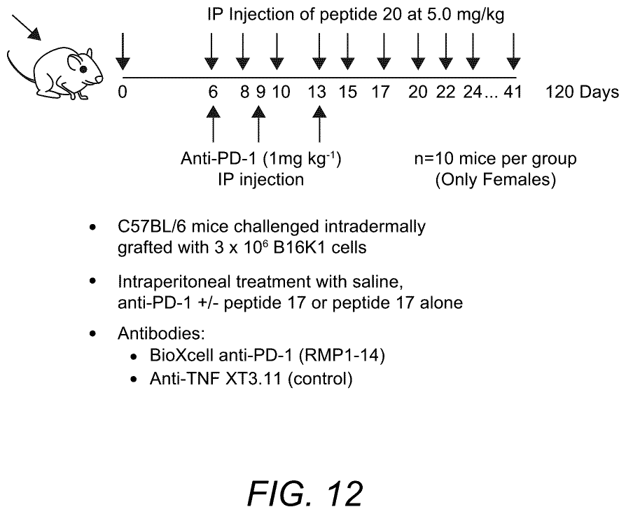

As shown in FIG. 12, C57BL/6 wild-type (WT) mice were intradermally grafted with 3×10⁶ B16K1 melanoma cells, followed by intraperitoneal (IP) administration of peptide 17 (SEQ ID NO. 17) (5 mg/kg) on days 6, 8, 10, 13, 15, 17, 20, 22, 24, and 41 (n=10 mice per group), an anti-PD-1 monoclonal antibody (BioXcell RMP-1-14) at 1 mg/kg on days 6, 9, and 13, peptide 17 (SEQ ID NO. 17) and anti-PD-1 monoclonal antibody on corresponding schedules, or anti-PD-1 monoclonal in combination with a 10 mg/kg of an anti-TNF monoclonal antibody (XT3.11, positive control). Mice were injected with saline as a negative control. Results are shown in FIG. 13.

FIG. 13 shows cumulative survival curves resulting from the indicated treatments as shown in FIG. 12. As shown, treatment with anti-PD-1 antibody alone is not distinguishable from saline. Similarly, treatment with peptide 17 (SEQ ID NO. 17) alone had no discernible effect (data not shown). In contrast, combination therapy with peptide 17 (SEQ ID NO. 17) and anti-PD-1 monoclonal antibody (at 1 mg/kg) was effective at extending survival. Other studies showed that this anti-PD-1 monoclonal antibody is effective at extending survival in a melanoma model when used at 10 mg/kg. Similar activity is expected for peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and/or peptide 33 (SEQ ID NO. 33).

It is believed that cyclic octadecapeptides (e.g., peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and/or peptide 33 (SEQ ID NO. 33)) can provide a less toxic and potentially more effective treatment in a broad range of oncologic indications in which suppression of MDSCs is a treatment goal.

### Cancer

The disclosed methods may be used to treat or prevent cancer, including metastatic cancer. Cancer is a group of related diseases that may include sustained proliferative signaling, evasion of growth suppressors, resistance to cell death, enablement of replicative immortality, induction of angiogenesis, and the activation of invasion and metastasis. The disclosed methods may enhance or elicits an immune response against a cancer in the subject. The immune response may lead to an increase in one or more of leukocytes, lymphocytes, monocytes, and eosinophils.

Cancer that may be treated by the disclosed methods, includes, but is not limited to, astrocytoma, adrenocortical carcinoma, appendix cancer, basal cell carcinoma, bile duct cancer, bladder cancer, bone cancer, brain cancer, brain stem cancer, brain stem glioma, breast cancer, cervical cancer, colon cancer, colorectal cancer, cutaneous T-cell lymphoma, diffuse intrinsic pontine glioma, ductal cancer, endometrial cancer, ependymoma, Ewing's sarcoma, esophageal cancer, eye cancer, fibrosarcoma, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, soft tissue carcinoma, soft tissue sarcoma, solid tumor, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer, and Wilms tumor.

In some embodiments, the cancer that may be treated by the disclosed methods is melanoma, renal cancer, prostate cancer, ovarian cancer, breast cancer, glioma, lung cancer, soft tissue carcinoma, soft tissue sarcoma, osteosarcoma, or pancreatic cancer. In some embodiments, the cancer is a solid tumor. In some embodiments, the cancer is a soft tissue carcinoma. In some embodiments, the cancer is a fibrosarcoma. In some embodiments, the cancer is diffuse intrinsic pontine glioma. In some embodiments, the cancer is a metastatic cancer.

### Pharmaceutical Compositions

In some embodiments, the cyclic peptides described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

It should be understood that in addition to the ingredients particularly mentioned above, the compounds and compositions described herein may include other agents conventional in the art having regard to the type of formulation in question, for example those suitable for oral administration may include flavoring agents.

Embodiments of the inventing concept include an immune checkpoint inhibitor and a cyclic peptide that is effective in enhancing the effects of the immune checkpoint inhibitor in treating cancer. In some embodiments such a kit can include the immune checkpoint inhibitor and the cyclic peptide in a comingled form (e.g., the same formulation). In other embodiments such a kit can provide the immune checkpoint inhibitor and the cyclic peptide as separate formulations. Such kits can include instructions for use. Such instructions can, for example, provide a treatment protocol in which effective amounts of an effective cyclic peptide are provided to a person in need of treatment for cancer in combination with an immune checkpoint inhibitor, In some embodiments the cyclic peptide and the immune checkpoint inhibitor are administered on the same schedule, and can be co-administered. In other embodiments the cyclic peptide and the immune checkpoint inhibitor are administered on different schedules. Such different schedules can be provide a period of time over which both the cyclic peptide and the immune checkpoint inhibitor are administered. Alternatively, such different schedules can provide a period of time over which either the cyclic peptide is administered or the immune checkpoint inhibitor is administered. Such schedules can provide the cyclic peptide and/or the immune checkpoint inhibitor continuously (e.g., by infusion) or periodically. A periodic schedule can provide the cyclic peptide and/or the immune checkpoint inhibitor once an hour, every two hours, every three hours, every four hours, four times a day, three times a day, twice a day, once daily, every two days, every three days, once a week, every two weeks, every three weeks, once a month, every two months, every three months, every four months, twice a year, annually, or at longer intervals.

While the data provided above is from animal models of melanoma and colon cancer, of the combinations described herein may also be effective in treating pancreatic cancer, aggressive lymphoma, or metastatic cancers. In view of the data, it would be expected that cyclic peptides (such as, but not limited to, peptide 1 (SEQ ID NO. 1), peptide 2 (SEQ ID NO. 2), peptide 3) will have utility that extends to a broad range of cancers and stages beyond the scope of the current data.

It should be apparent to those skilled in the art that many more modifications besides those already described are possible without departing from the disclosure provided herein. The disclosed subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

What is claimed is:

1. A method of treating cancer in a patient in need thereof, comprising administering to a patient an effective amount of a cyclic peptide and an effective amount of an immune checkpoint inhibitor (ICI).

2. The method of claim, 1, wherein the cyclic peptide is an analog of a θ-defensin.

3. The method of claim 2, wherein the cyclic peptide is selected from the group consisting of peptide 1 (SEQ ID NO. 1), peptide 2 (SEQ ID NO. 2), peptide 3 (SEQ ID NO. 3), peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16).

4. The method of claim 1, wherein the cyclic peptide is a naturally occurring θ-defensin.

5. The method of claim 1, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

6. The method of one of claims 1 to 5, wherein treating cancer comprises inducing tumor regression.

7. The method of claim 6, wherein tumor regression is induced in melanoma.

8. The method of one of claims 1 to 7, wherein treating cancer comprises preventing tumor reoccurrence.

9. The method of claim 8, wherein tumor reoccurrence is prevented for melanoma or colon cancer.

10. The method of one of claims 1 to 9, wherein treating cancer comprises suppressing myeloid-derived suppressor cell infiltration into a tumor microenvironment.

11. The method of one of claims 1 to 10, wherein treating cancer comprises augmenting CD8+ T cell responses in the tumor microenvironment when used in combination with the ICI.

12. The method of one of claims 1 to 11, wherein the ICI comprises an antibody directed to PD-1.

13. The method of one of claims 1 to 12, wherein treating cancer comprises reducing dosage of the ICI relative to the amount of ICI administered without the peptide while retaining effectiveness.

14. The method of claim 13, wherein treating cancer further comprises reducing or eliminating side effects associated with administration of the ICI.

15. The method of one of claims 1 to 14, wherein the effective amount of the cyclic peptide is from 0.01 mg/kg to 15 mg/kg.

16. The method of one of claims 1 to 15, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

17. The method of claim 16, wherein the ICI is an anti-PD-1 antibody.

18. A method of enhancing immune checkpoint inhibitor therapy, comprising administering an effective amount of a cyclic peptide in combination with an immune checkpoint inhibitor (ICI).

19. The method of claim 18, wherein the cyclic peptide is an analog of a theta defensin.

20. The method of claim 19, wherein the cyclic peptide is selected from the group consisting of peptide 1 (SEQ ID NO. 1), peptide 2 (SEQ ID NO. 2), and peptide 3 (SEQ ID NO. 3).

21. The method of claim 19, wherein the cyclic peptide is selected from the group consisting of peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16).

22. The method of claim 18, wherein the cyclic peptide is a naturally occurring θ-defensin.

23. The method of claim 22, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

24. The method of one of claims 16 to 21, wherein the immune checkpoint inhibitor is an antibody directed to PD-1.

25. The method of one of claims 18 to 24, wherein enhancing immune checkpoint inhibitor therapy comprises reducing dosage of the ICI relative to the amount of ICI administered without the peptide while retaining effectiveness.

26. The method of claim 25, wherein enhancing immune checkpoint inhibitor therapy further comprises reducing or eliminating side effects associated with administration of the ICI.

27. The method of one of claims 18 to 26, wherein the effective amount of the cyclic peptide is from 0.01 mg/kg to 15 mg/kg.

28. The method of one of claims 18 to 27, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

29. The method of claim 28, wherein the ICI is an anti-PD-1 antibody.

30. A pharmaceutical composition comprising a cyclic peptide in an amount effective to enhance an immune checkpoint therapy.

31. The pharmaceutical composition of claim 30, wherein the cyclic peptide is an analog of a 0-defensin.

32. The pharmaceutical composition of claim 31, wherein the cyclic peptide is selected from the group consisting of peptide 1 (SEQ ID NO. 1), peptide 2 (SEQ ID NO. 2), and peptide 3 (SEQ ID NO. 3).

33. The pharmaceutical composition of claim 31, wherein the cyclic peptide is selected from the group consisting of peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16).

34. The pharmaceutical composition of claim 27, wherein the cyclic peptide is a naturally occurring θ-defensin.

35. The pharmaceutical composition of claim 34, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

36. The pharmaceutical composition of one of claims 30 to 35, wherein the immune checkpoint therapy comprises administration of an ICI selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

37. The pharmaceutical composition of one of claims 30 to 36, further comprising an immune checkpoint inhibitor.

38. The pharmaceutical composition of claim 37, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

39. The pharmaceutical composition of one of claims 36 to 38, wherein the immune checkpoint inhibitor is an antibody to PD-1.

40. The pharmaceutical composition of one of claims 30 to 39, wherein the effective amount of the cyclic peptide is from 0.01 mg/kg to 15 mg/kg.

41. Use of a cyclic peptide in treating cancer in a patient, wherein an effective amount of a cyclic peptide is provided in combination with administration of an effective amount of an immune checkpoint inhibitor (ICI).

42. The use of claim 41, wherein the cyclic peptide is an analog of a θ-defensin.

43. The use of claim 42, wherein the cyclic peptide is selected from the group consisting of peptide 1 (SEQ ID NO. 1), peptide 2 (SEQ ID NO. 2), and peptide 3 (SEQ ID NO. 3).

44. The use of claim 42, wherein the cyclic peptide is selected from the group consisting of peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), ), peptide 13 (SEQ ID NO. 13), peptide 14 (SEQ ID NO. 14), peptide 15 (SEQ ID NO. 15), and peptide 16 (SEQ ID NO. 16).

45. The use of claim 41, wherein the cyclic peptide is a naturally occurring θ-defensin.

46. The use of claim 45, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

47. The use of one of claims 41 to 46, wherein treating cancer comprises inducting tumor regression.

48. The use of claim 47, wherein tumor regression is induced in melanoma.

49. The use of one of claims 41 to 48, wherein treating cancer comprises preventing tumor reoccurrence.

50. The use of claim 49, wherein tumor reoccurrence is prevented for melanoma or colon cancer.

51. The use of one of claims 41 to 50, wherein treating cancer comprises suppressing myeloid-derived suppressor cell infiltration into a tumor microenvironment.

52. The use of one of claims 41 to 51, wherein treating cancer comprises augmenting CD8+ T cell responses in the tumor microenvironment when used in combination with the ICI.

53. The use of one of claims 41 to 52, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

54. The use of one of claims 41 to 52, wherein the ICI comprises an antibody directed to PD-1.

55. The use of one of claims 41 to 54, wherein treating cancer comprises reducing dosage of the ICI relative to the amount of ICI administered without the peptide while retaining effectiveness.

56. The use of claim 55, wherein treating cancer further comprises reducing or eliminating side effects associated with administration of the ICI.

57. The use of one of claims 41 to 56, wherein the effective amount of the cyclic peptide is from 0.01 mg/kg to 15 mg/kg.

58. Use of a cyclic peptide to enhance immune checkpoint inhibitor therapy, wherein an effective amount of a cyclic peptide is provided in combination with an immune checkpoint inhibitor (ICI).

59. The use of claim 58, wherein the cyclic peptide is an analog of a θ-defensin.

60. The use of claim 59, wherein the cyclic peptide is selected from the group consisting of peptide 1 (SEQ ID NO. 1), peptide 2 (SEQ ID NO. 2), and peptide 3 (SEQ ID NO. 3).

61. The use of claim 59, wherein the cyclic peptide is selected from the group consisting of peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), peptide 13 (SEQ ID NO. 13), peptide 14 (SEQ ID NO. 14), peptide 15 (SEQ ID NO. 15), and peptide 16 (SEQ ID NO. 16).

62. The use of claim 58, wherein the cyclic peptide is a naturally occurring θ-defensin.

63. The use of claim 62, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

64. The use of one of claims 58 to 63, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

65. The use of one of claims 58 to 64, wherein the immune checkpoint inhibitor is an antibody directed to PD-1.

66. The use of one of claims 58 to 65, wherein enhancing comprises reducing dosage of the ICI relative to the amount of ICI administered without the peptide while retaining effectiveness.

67. The use of claim 77, wherein enhancing further comprises reducing or eliminating of side effects associated with administration of the ICI.

68. The use of one of claims 58 to 67, wherein the effective amount of the synthetic cyclic peptide is from 0.01 mg/kg to 15 mg/kg.

69. A synthetic cyclic peptide consisting of peptide 2 (SEQ ID NO. 2) and having the following structure:

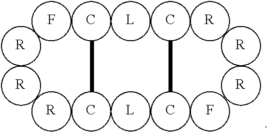

70. A pharmaceutical composition comprising peptide 2 and a pharmaceutically acceptable excipient.

71. A kit comprising:
a cyclic peptide; and
an immune checkpoint inhibitor.

72. The kit of claim 71, wherein the cyclic peptide is an analog of a θ-defensin.

73. The kit of claim 72, wherein the cyclic peptide is selected from the group consisting of peptide 1 (SEQ ID NO. 1), peptide 2 (SEQ ID NO. 2), and peptide 3 (SEQ ID NO. 3).

74. The kit of claim 72, wherein the cyclic peptide is selected from the group consisting of peptide 4 (SEQ ID NO. 4), peptide 5 (SEQ ID NO. 5), peptide 6 (SEQ ID NO. 6), peptide 7 (SEQ ID NO. 7), peptide 8 (SEQ ID NO. 8), peptide 9 (SEQ ID NO. 9), peptide 10 (SEQ ID NO. 10), peptide 11 (SEQ ID NO. 11), peptide 12 (SEQ ID NO. 12), ), peptide 13 (SEQ ID NO. 13), peptide 14 (SEQ ID NO. 14), peptide 15 (SEQ ID NO. 15), and peptide 16 (SEQ ID NO. 16).

75. The kit of claim 71, wherein the cyclic peptide is a naturally occurring θ-defensin.

76. The use of claim 75, wherein the cyclic peptide is selected from the group consisting of peptide 17 (SEQ ID NO. 17), peptide 18 (SEQ ID NO. 18), peptide 19 (SEQ ID NO. 19), peptide 20 (SEQ ID NO. 20), peptide 21 (SEQ ID NO. 21), peptide 22 (SEQ ID NO. 22), peptide 23 (SEQ ID NO. 23), peptide 24 (SEQ ID NO. 24), peptide 25 (SEQ ID NO. 25), peptide 26 (SEQ ID NO. 26), peptide 27 (SEQ ID NO. 27), peptide 28 (SEQ ID NO. 28), peptide 29 (SEQ ID NO. 29), peptide 30 (SEQ ID NO. 30), peptide 31 (SEQ ID NO. 31), peptide 32 (SEQ ID NO. 32), and peptide 33 (SEQ ID NO. 33).

77. The kit of one of claims 71 to 76, wherein the cyclic peptide is provided in an amount effective to enhance activity of the immune checkpoint inhibitor.

78. The kit of one of claims 71 to 77, wherein the cyclic peptide and the immune checkpoint inhibitor are comingled.

79. The kit of one of claims 71 to 78, wherein the ICI is selected from the group consisting of an anti-natural killer cell receptor 2B4 (2B4), anti-B- and T-lymphocyte attenuator (BTLA), anti-CD160 antigen (CD160), anti-cytotoxic T-lymphocyte protein 4 (CTLA-4), anti-lymphocyte activation gene 3 protein (LAG-3), anti-programmed cell death protein 1 (PD-1), anti-T-cell immunoglobulin mucin receptor 3 (TIM-3), and an anti-T-cell immunoreceptor with Ig and ITIM domains (TIGIT) agents/inhibitor.

80. The kit of one of claims 71 to 79, wherein the immune checkpoint inhibitor is an anti-PD-1 monoclonal antibody.

81. The kit of one of claims 71 to 80, comprising instructions for use of the cyclic peptide in combination with the immune checkpoint inhibitor to treat cancer.


SEQUENCE LISTING (33 sequences)

SEQ ID NO: 1 - protein, 14 aa, synthetic construct
CICRRRFCLCRRGV

SEQ ID NO: 2 - protein, 14 aa, synthetic construct
CLCRRRFCLCRRRF

SEQ ID NO: 3 - protein, 14 aa, synthetic construct
CICRRRFCLCRRGA

SEQ ID NO: 4 - protein, 14 aa, synthetic construct
CSCRRRFCLCRRGV

SEQ ID NO: 5 - protein, 14 aa, synthetic construct
CICRRGVCICRRGV

SEQ ID NO: 6 - protein, 14 aa, synthetic construct
CSCRRRFCICRRGV

SEQ ID NO: 7 - protein, 14 aa, synthetic construct
CLCRRGVCLCRRGV

SEQ ID NO: 8 - protein, 14 aa, synthetic construct
CICRRRFCSCRRGV

SEQ ID NO: 9 - protein, 14 aa, synthetic construct
CICRRRVCICGRGV

SEQ ID NO: 10 - protein, 14 aa, synthetic construct
CACARRFCACRRGV

SEQ ID NO: 11 - protein, 14 aa, synthetic construct
CICRRRVCICRRGV

SEQ ID NO: 12 - protein, 14 aa, synthetic construct
CICRRRACLCRRGL

SEQ ID NO: 13 - protein, 14 aa, synthetic construct
CRCRRGVCRCRRGV

SEQ ID NO: 14 - protein, 14 aa, synthetic construct
GFCRCRRGVCRCTR

SEQ ID NO: 15 - protein, 14 aa, synthetic construct
GVCIVRRRFCLCRR

SEQ ID NO: 16 - protein, 14 aa, synthetic construct
GVCLCIRGRCRCRR

SEQ ID NO: 17 - protein, 18 aa, Macaca mulatta
GFCRCLCRRGVCRCICTR

SEQ ID NO: 18 - protein, 18 aa, Macaca mulatta
GVCRCLCRRGVCRCLCRR

SEQ ID NO: 19 - protein, 18 aa, Macaca mulatta
GFCRCICTRGFCRCICTR

SEQ ID NO: 20 - protein, 18 aa, Macaca mulatta
GICRCICTRGFCRCICVL

SEQ ID NO: 21 - protein, 18 aa, Macaca mulatta
GICRCLCRRGVCRCICVL

SEQ ID NO: 22 - protein, 18 aa, Macaca mulatta
GICRCICVLGICRCICVL

SEQ ID NO: 23 - protein, 18 aa, Papio anubis
CVCRRGVCRCVCTRGFCR

SEQ ID NO: 24 - protein, 18 aa, Papio anubis
CVCRRGVCRCVCRRGVCR

SEQ ID NO: 25 - protein, 18 aa, Papio anubis
CICLLGICRCVCTRGFCR

SEQ ID NO: 26 - protein, 18 aa, Papio anubis
CICLLGICRCVCRRGFCR

SEQ ID NO: 27 - protein, 18 aa, Papio anubis
CICLLGICRCVCRRGVCR

SEQ ID NO: 28 - protein, 18 aa, Papio anubis
CICLLGICRCICLLGICR

SEQ ID NO: 29 - protein, 18 aa, Papio anubis
CFCRRGVCRCVCTRGFCR

SEQ ID NO: 30 - protein, 18 aa, Papio anubis
CFCRRGVCRCVCRRGVCR

SEQ ID NO: 31 - protein, 18 aa, Papio anubis
CFCRRGVCRCICLLGICR

SEQ ID NO: 32 - protein, 18 aa, Papio anubis
CFCRRGVCRCFCRRGVCR

SEQ ID NO: 33 - protein, 18 aa, Homo sapiens
CFCRRGVCRCFCRRGVCR